SEQUENCE LISTING (5 sequences)

SEQ ID NO: 1 - protein, 824 aa, Artificial Sequence
  synthetic protein ofr HSV2 ICP0 with RING domain
MEPRPGTSSRADPGPERPPRQTPGTPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPPLRCQSFPCLHPFCIPCMKTWIPLRNTCPLCNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 2 - protein, 783 aa, Artificial Sequence
  synthetic protein of HSV2 ICP0 Mutant #1 (mICP0)
MEPRPGTSSRADPGPERPPRQTPGTPAAPHAWGMLNDMQWLASSDSEEETEVGISDDDLHRDSTSEAGSTDTEMFEAGLMDAATPPARPPAERQGSPTPADAQGSCGGGPVGEEEAEAGGGGDVCAVCTDEIAPLTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 3 - protein, 750 aa, Artificial Sequence
  synthetic protein of HSV1 ICP0 Mutant #2 (m2ICP0)
HRDSTSEAGSTDTEMFEAGLMDAATPPARPPAERQGSPTPADAQGSCGGGPVGEEEAEAGGGGDVCAVCTDEIAPPLRCQSFPCLHPFCIPNTPVAYLIVGVTASGSFSTIPIVNDPRTRVEAEAAVRAGTAVDFIWTGNPRTAPRSLSLGGHTVRALSPTPPWPGTDDEDDDLADVDYVPPAPRRAPRRGGGGAGATRGTSQPAATRPAPPGAPRSSSSGGAPLRAGVGSGSGGGPAVAAVVPRVASLPPAAGGGRAQARRVGEDAAAAEGRTPPARQPRAAQEPPIVISDSPPPSPRRPAGPGPLSFVSSSSAQVSSGPGGGGLPQSSGRAARPRAAVAPRVRSPPRAAAAPVVSASADAAGPAPPAVPVDAHRAPRSRMTQAQTDTQAQSLGRAGATDARGSGGPGAEGGPGVPRGTNTPGAAPHAAEGAAARPRKRRGSDSGPAASSSASSSAAPRSPLAPQGVGAKRAAPRRAPDSDSGDRGHGPLAPASAGAAPPSASPSSQAAVAAASSSSASSSSASSSSASSSSASSSSASSSSASSSSASSSAGGAGGSVASASGAGERRETSLGPRAAAPRGPRKCARKTRHAEGGPEPGARDPAPGLTRYLPIAGVSSVVALAPYVNKTVTGDCLPVLDMETGHIGAYVVLVDQTGNVADLLRAAAPAWSRRTLLPEHARNCVRPPDYPTPPASEWNSLWMTPVGNMLFDQGTLVGALDFHGLRSRHPWSREQGAPAPAGDAPAGHGE

SEQ ID NO: 4 - protein, 393 aa, Artificial Sequence
  synthetic protein of HSV2 glycoprotein D for Ad-gD-Luc construct
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 5 - protein, 107 aa, Artificial Sequence
  synthetic protein of RING domain
  MISC_FEATURE (2)..(3): Xaa can be any amino acid
  MISC_FEATURE (5)..(9): Xaa can be any amino acid
  MISC_FEATURE (10)..(43): Xaa can be any amino acid or absent
  MISC_FEATURE (45)..(45): Xaa can be any amino acid
  MISC_FEATURE (46)..(47): Xaa can be any amino acid or absent
  MISC_FEATURE (49)..(50): Xaa can be any amino acid
  MISC_FEATURE (51)..(51): Xaa can be any amino acid or absent
  MISC_FEATURE (52)..(52): Xaa can be Asn, Cys or His
  MISC_FEATURE (53)..(54): Xaa can be any amino acid
  MISC_FEATURE (56)..(59): Xaa can be any amino acid
  MISC_FEATURE (60)..(103): Xaa can be any amino acid or absent
  MISC_FEATURE (105)..(106): Xaa can be any amino acid
CXXCXXXXXXXXXXXXXXXXXXXXXXXXXXXXXXXXXXXXXXXCXXXHXXXXXXCXXXXXXXXXXXXXXXXXXXXXXXXXXXXXXXXXXXXXXXXXXXXXXXXCXXC

(19) United States
(12) Patent Application Publication
(10) Pub. No.: US 2017/0298389 A1
(43) Pub. Date: Oct. 19, 2017

(54) HSV VACCINES

(71) Applicant: Vaxart, Inc., South San Francisco, CA (US)

(72) Inventors: Ciaran Scallan, San Francisco, CA (US); Wendy Peters, South San Francisco, CA (US)

(73) Assignee: Vaxart, Inc., South San Francisco, CA (US)

(21) Appl. No.: 15/517,768

(22) PCT Filed: Oct. 9, 2015
(86) PCT No.: PCT/US15/54929

§ 371 Date: Apr. 7, 2017

Related U.S. Application Data
(60) Provisional application No. 62/062,692, filed on Oct. 10, 2014.

(51) Int. Cl. C12N 15/869; A61K 39/245; C07K 14/035; C12N 15/09; C12N 7/04; C07K 14/005; A61K 39/00; C12N 15/00

(52) U.S. Cl. CPC C12N 15/869; C12N 7/04; A61K 39/245; C07K 14/005; C07K 14/035; C12N 15/09; C12N 15/00; A61K 39/00; C12N 2710/16622; C12N 2710/16643

(57) ABSTRACT

Provided herein are improved vaccines for HSV-2.

### BACKGROUND OF THE INVENTION

Herpes Simplex Virus 2 (HSV-2) infects epithelial cells of the genital mucosa and can migrate to neurons (autonomic ganglia) where it remains dormant. When it comes out of dormancy, it causes painful genital lesions. HSV-2, and closely related HSV-1, are complex viruses and can circumvent and misdirect the immune system of its host.

Efforts at developing a vaccine for HSV-2 have been unsuccessful. Whole, inactivated virus, attenuated live virus, modified live virus, and cell culture-derived subunits were largely unsuccessful or had low efficacy. Vaccines comprised of one or two of the envelope glycoproteins (gD or gD and gB) in combination with adjuvants (MF59 or MPL1 and alum) have also been attempted. The glycoproteins were attractive candidates mainly because they are the targets of neutralizing antibodies and are highly conserved among HSV-2 strains, yet these efforts were discontinued due to lack of success. The glycoproteins do not elicit a strong CD8 T cell response, important for eliminating virally infected cells and controlling HSV-2 outbreaks. The lack of efficacy may also be because injected vaccines do not elicit substantial mucosal T cell responses.

Current treatments of HSV-2 lesions include daily treatment with Valtrex®, Zovirax®, or Famvir®. Even with these treatments, however, the infected individual can have outbreaks and shed virus.

### BRIEF SUMMARY OF THE INVENTION

Provided herein are methods and compositions that elicit substantial mucosal T cell responses specific for HSV. The presently disclosed compositions can be administered orally or mucosally (e.g., vaginally) to result in better compliance than injectable vaccines or treatments. The presently disclosed compositions function prophylactically, to reduce the likelihood of HSV infection of a non-infected individual, or to reduce symptoms in an infected individual, e.g., number of outbreaks, severity of lesions, HSV shedding, and risk of spreading the virus to partners.

Provided herein are pharmaceutical compositions, e.g., HSV vaccines, comprising an ICP0 antigen, wherein the ICP0 antigen has a mutation in the RING domain compared to wild type HSV ICP0 (e.g., a substitution, insertion or deletion in the RING domain of HSV ICP0). In some embodiments, the ICP0 antigen has a mutation in at least one of the conserved amino acids of the RING domain compared to the wild type ICP0 polypeptide. In some embodiments, the HSV is HSV-2. An exemplary wild type HSV-2 ICP0 sequence is shown in SEQ ID NO:1. In some embodiments, pharmaceutical composition is formulated for injection. In some embodiments, the pharmaceutical composition is formulated for oral, mucosal, or vaginal administration.

In some embodiments, the ICP0 antigen retains at least one T cell epitope from HSV-2 ICP0, e.g., at least 2, 3, 4, 5, 6, 7, 8 or more T cell epitopes from HSV-2 ICP0. In some embodiments, the at least one T cell epitope is independently selected from the group consisting of amino acids 83-89; amino acids 124-150; amino acids 214-222; amino acids 636-662; amino acids 693-701; amino acids 720-729; amino acids 741-751; and amino acids 783-792 in any combination.

In some embodiments, the ICP0 antigen comprises a polypeptide with at least 80% identity (e.g., 85, 90, 91, 92, 93, 94, 95, 96, 97, 98, 99, or 100% identity) to the sequence of SEQ ID NO:2. In some embodiments, the ICP0 antigen comprises a polypeptide with at least 80% identity (e.g., 85, 90, 91, 92, 93, 94, 95, 96, 97, 98, 99, or 100% identity) to the sequence of SEQ ID NO:3.

In some embodiments, the pharmaceutical composition further comprises dsRNA or a dsRNA mimetic. In some embodiments, the pharmaceutical composition further comprises at least one HSV capsid, envelope, or tegument protein, or mutants thereof. In some embodiments, the HSV protein is glycoprotein B or glycoprotein D. In some embodiments, the pharmaceutical composition further comprises at least one regulatory protein such as ICP4 or ICP10, or mutants thereof.

Provided herein are expression vectors comprising a promoter operably linked to a polynucleotide encoding an ICP0 antigen, wherein the ICP0 antigen has a mutation in the RING domain compared to wild type HSV ICP0 (e.g., a substitution, insertion or deletion in the RING domain of HSV ICP0). In some embodiments, the expression vector is a viral vector, e.g., an adenoviral vector, or a plasmid. In some embodiments, the ICP0 antigen has a mutation in at least one of the conserved amino acids of the RING domain compared to the wild type ICP0 polypeptide. In some embodiments, the HSV is HSV-2.

In some embodiments, the ICP0 antigen retains at least one T cell epitope from HSV-2 ICP0, e.g., at least 2, 3, 4, 5, 6, 7, 8 or more T cell epitopes from HSV-2 ICP0. In some embodiments, the at least one T cell epitope is independently selected from the group consisting of amino acids 83-89; amino acids 124-150; amino acids 214-222; amino acids 636-662; amino acids 693-701; amino acids 720-729; amino acids 741-751; and amino acids 783-792 in any combination.

In some embodiments, the ICP0 antigen comprises a polypeptide with at least 80% identity (e.g., 85, 90, 91, 92, 93, 94, 95, 96, 97, 98, 99, or 100% identity) to the sequence of SEQ ID NO:2. In some embodiments, the ICP0 antigen comprises a polypeptide with at least 80% identity (e.g., 85, 90, 91, 92, 93, 94, 95, 96, 97, 98, 99, or 100%/0 identity) to the sequence of SEQ ID NO:3.

In some embodiments, the expression vector (e.g., adenoviral vector) further comprises a promoter operably linked to polynucleotide encoding dsRNA. In some embodiments, the expression vector further comprises a promoter operably linked to a polynucleotide encoding an HSV capsid, envelope, tegument, or regulatory protein or a mutant thereof. In some embodiments, the expression vector further comprises both a polynucleotide encoding dsRNA and a polynucleotide encoding an HSV capsid, envelope, tegument, or regulatory protein or a mutant thereof. In some embodiments, the HSV protein is gD, gB, ICP4 or ICP10.

Further provided are pharmaceutical compositions, e.g., HSV vaccines, comprising the expression vector described above. In some embodiments, the pharmaceutical composition is formulated for oral or mucosal administration. In some embodiments, the pharmaceutical composition is formulated for administration by injection. In some embodiments, the pharmaceutical composition further comprises dsRNA or a dsRNA mimetic. In some embodiments, the pharmaceutical composition further comprises a second expression vector (e.g., viral or adenoviral vector, or plasmid), wherein the second expression vector comprises polynucleotide encoding an HSV (e.g., HSV-2) capsid, envelope, tegument, or regulatory protein, or mutants thereof, optionally operably linked to a promoter. In some embodiments, the pharmaceutical composition further comprises a third expression vector comprising a polynucleotide encoding an additional HSV (e.g., HSV-2) capsid, envelope, tegument, or regulatory protein, or mutants thereof, optionally operably linked to a promoter. In some embodiments, the HSV protein is HSV-2 gD. In some embodiments, the HSV protein is HSV-2 gB. In some embodiments, the HSV protein is HSV-2 ICP4 or ICP10.

Also provided are methods for eliciting an immune response in an individual comprising administering any of the pharmaceutical compositions described herein to the individual. In some embodiments, the administration causes a cytotoxic T cell (CD8+ T cell) response in the individual. In some embodiments, the cytotoxic T cells are specific for ICP0 antigen. In some embodiments, the administration is oral. In some embodiments, the administration is vaginal. In some embodiments, the administration is mucosal. In some embodiments, the administration is by injection (intramuscular, intraperitoneal, intravenous, subcutaneous, etc.). In some embodiments, the administration is periodic (e.g., weekly, monthly, yearly), or episodic (e.g., before or after potential exposure to HSV, or when lesions arise). In some embodiments, the individual has HSV-2. In some embodiments, the individual has not been diagnosed with HSV-2 infection.

Provided herein are methods for reducing an HSV (e.g., HSV-2) symptom in an individual infected with HSV-2 comprising administering the pharmaceutical compositions described herein to the individual. In some embodiments, the administration reduces the HSV symptom by at least 5% (e.g., at least 10%, 15%, 20%, 25%, 30%, 35%, 40%, 50%, 60%, 70%, 75%, 80%, 90%, 95%, or 100%) compared to the HSV symptom prior to administration. In some embodiments, the HSV symptom is frequency of outbreak. In some embodiments, the HSV symptom is severity of lesion. In some embodiments, the HSV symptom is amount of viral shedding. In some embodiments, the administration is oral. In some embodiments, the administration is vaginal. In some embodiments, the administration is mucosal. In some embodiments, the administration is by injection (intramuscular, intraperitoneal, intravenous, subcutaneous, etc.). In some embodiments, the administration is periodic (e.g., weekly, monthly, yearly), or episodic (e.g., before or after potential exposure to HSV, or when lesions arise).

Additionally provided are methods of vaccinating an uninfected individual, or an individual that has not been diagnosed with HSV, against HSV (e.g., HSV-2), comprising administering any of the pharmaceutical compositions described herein. In some embodiments, the administration is oral. In some embodiments, the administration is vaginal. In some embodiments, the administration is mucosal. In some embodiments, the administration is by injection (intramuscular, intraperitoneal, intravenous, subcutaneous, etc.). In some embodiments, the administration is periodic (e.g., weekly, monthly, yearly), or episodic (e.g., before or after potential exposure to HSV).

### BRIEF DESCRIPTION OF THE DRAWINGS

FIG. 1 outlines the structural elements of a chimeric adenoviral vector for HSV-2 vaccination. FIG. 1A is a schematic of the constructs described in more detail in Example 1. CMV=cytomegalovirus promoter; BGH PA=bovine growth hormone poly-A tail; SPA=synthetic poly-A tail. FIG. 1B shows the wild type ICP0 polypeptide sequence (SEQ ID NO:1) with the RING domain and deletions indicated. FIGS. 1C and 1D show the sequences of the resulting mutant polypeptides. Mutant #1 (mICP0, SEQ ID NO:2) and Mutant #2 (m2ICP0, SEQ ID NO:3), respectively. FIG. 1E shows the wild type ICP0 polypeptide sequence with CD8+ T cell epitopes identified in humans highlighted. The RING domain and m2ICP0 deletion are also indicated. FIG. 1F shows the sequence of the Glycoprotein D polypeptide (SEQ ID NO:4).

FIG. 2. In vitro expression of Ad-HSV2 vaccine constructs. RNA copy numbers were determined post infection for wild-type (wICP0) and mutant ICP0 (mICP0 & m2ICP0) vaccine vectors by RT-QPCR (FIG. 2A). An Ad-HSV2 VP22 vector was included as a negative control. ICP0 protein levels were evaluated by Western blot analysis of infected cell lysates run under reducing conditions (FIG. 2B). Glycoprotein D expression was confirmed by Western blot analysis for the Ad-CMV-gD construct (FIG. 2C).

FIG. 3. Immunization with wild type ICP0 (wICP0). Mutant #1 (mICP0), or Mutant #2 (m2ICP0) induces T cell responses, as measured by interferon gamma (IFN-γ) release. FIG. 3A shows that the average mICP0 IFN-γ spot forming cell count was higher than that of wIPC0 in spleens following intramuscular immunization. FIG. 3B shows that m2ICP0 IFN-γ spot forming cell count in iliac lymph nodes was slightly higher than that of mlPCO. In FIG. 3B, an IFN-γ Elispot was performed using single cell suspensions from ILNs. Balb/c mice were vaccinated 3 times intravaginally. ILN were harvested 1 week after final vaccination n=10 and 12 mice (p=0.3 by Mann Whitney).

FIG. 4. Both gD (glycoprotein D) and ICP0 antigens elicit T cell responses. Animals were co-immunized with gD and mICP0 constructs 3 times by vaginal administration, 1 week apart, and the T cell responses to gD and ICP0 measured 1 week after the final administration by IFN-γ ELISpot. FIG. 4A shows IFN-γ response in the spleen, and FIG. 4B shows IFN-γ response in iliac lymph nodes following immunization with gD peptide pool (black bars); ICP0 peptide pool (grey bars); or unstimulated (open bars). Animals 7, 8, and 9 were untreated.

FIG. 5. CD8 T cells are recruited to the genital tract after immunization. Mice were immunized by vaginal administration 3 times, 1 week apart. One week after the final vaccination genital tracts were harvested. Mononuclear cells from individual mouse genital tracts were isolated after tissue digestion, and T cells were quantitated by flow cytometry. FIG. 5A shows the increase in CD8+ T cells as a percentage of T cells in the genital tract in vaccinated mice compared to naïve, non-vaccinated mice. FIG. 5B illustrates the results as dot plots (CD4 vertical vs. CD8 horizontal).

FIG. 6. T cells isolated from the genital tract after immunization are antigen specific. IFN-γ T cell responses measured by ELISpot assay are shown for T cells isolated from the genital tracts of immunized mice (either pooled from 2 or 3 mice) or unimmunized naïve mice (pooled from 3 mice). As a positive control, a spleen from one immunized mouse was used (Spleen). T cells isolated from the genital tract (spleen) were either unstimulated (left, open bars), or immunized with gD peptide pool (middle, black bars) or ICP0 peptide pool (right, grey bars).

FIG. 7. Therapeutic challenge model in guinea pigs (GP). The test was performed on 4 different groups: 1. Oral vaccination with first adenoviral vector encoding gD and second adenoviral vector encoding ICP0 antigen; 2. Vaginal (iVag) vaccination with first adenoviral vector encoding gD and second adenoviral vector encoding mICP0 antigen; 3. Negative control (no vaccination); 4. Intramuscular (IM) vaccination with gD peptide antigen. Guinea pigs were scored 0-4: 0=negative; I=slight erythema (redness) or healing vesicles; 2=moderate erythema with swelling; 3=severe erythema with swelling and small vesicles; 4=severe erythema with swelling and large vesicles.

FIG. 8. Vaccination with rAD-gD-dsRNA reduces clinical scores in an HSV-2 therapeutic guinea pig model. Cumulative lesion scores are shown for individual guinea pigs between day 28 (last day of vaccination) and day 63 (termination day). Guinea pigs were treated intravaginally with PBS (negative control; filled black circles) or rAD-gD-dsRNA (open circles), or intramuscularly with gD protein plus ASO4 adjuvant (grey circles). Guinea pigs immunized vaginally with the adenoviral vector expressing gD had reduced clinical scores compared to the negative control animals, and had similar scores to the intramuscular positive control animals.

FIG. 9. Immunization with rAD-gD-dsRNA and rAD-mICP0-dsRNA mucosally provides clinical benefit in a therapeutic HSV-2 model. FIG. 9A shows the results of cumulative lesion scores from day 14-63 post-infection for the 4 groups (rAd-gD-dsRNA and rAd-mICP0-dsRNA delivered either vaginally or orally; gD protein+MPL/Alum (positive control) delivered intramuscularly; or non-immunized negative control). The oral and vaginal groups vaccinated with one adenoviral vector encoding gD and a second adenoviral vector encoding the mICP0 antigen showed reduced cumulative lesion scores compared to the negative control (top line). In addition the groups given the adenoviral vectors together trended toward having a reduced cumulative lesion score compared to the positive intramuscular control. FIG. 9B shows cumulative average lesion scores for later time points from the same study as shown in FIG. 8. Lesion scores were measured on days 33-63 (after the last immunization on day 28). Again, the adenoviral vaccines administered orally or vaginally demonstrate reduced clinical symptom scores compared to the negative control or the intramuscular positive control.

FIG. 10. Pre-infection vaccination acts prophylactically for significant protection against lesions. Vaccination with both the adenoviral vector encoding gD and the adenoviral vector encoding the mICP0 antigen were administered on days 0, 7 and 14, and guinea pigs were infected 2 weeks later on day 28 with HSV-2. The results show significantly reduced lesions in vaccinated animals (bottom line, diamonds) compared to negative control (top line, squares).

### DETAILED DESCRIPTION OF THE INVENTION

### I. Introduction

Previous attempts at developing a vaccine or treatment for HSV-2 have been based on envelope proteins of the virus. These do not result in a strong mucosal CD8 T cell response, which can clear virally infected cells. The presently disclosed vaccines are based on ICP0, an immediate early gene that plays a role in role in regulating both viral and host genes necessary for host cell infection and viral replication, as well as activating latent integrated virus. ICP0 also suppresses the host immune responses, e.g., by preventing activation of NFkB by Toll Like Receptors (TLRs) which are part of the innate immune defense against viruses (van Lint et al. (2010) J. Virol. 84:10802). ICP0 inhibits activation of interferons by IRF; interferon signaling is integral to the detection and elimination of viruses by the immune system (Paladino et al. (2010) PLoS One 5:E10428). While ICP0 functions to promote viral replication and activation, its early expression and multi-functional pro-viral role makes it a prime target as a T cell antigen.

Provided herein are mutants of ICP0 that reduce its pro-viral activity while retaining T cell epitopes, thereby providing a superior antigen for immune education and vaccination. These ICP0 antigens can be encoded on an adenoviral vector, optionally in combination with dsRNA, which is believed to act through TLR3 and/or IRF. Unlike naturally occurring ICP0, the presently disclosed ICP0 antigens do not significantly interfere with TLR3 or IRF activity.

The results shown herein demonstrate that immunization with an envelope protein (glycoprotein D (gD)) or ICP0 antigen result in homing of CD8 T cells to the genital mucosa. The majority of these cells bind integrin α4β7, which binds the mucosal epithelia via MAdCAM01. Immunization with a viral vector encoding ICP0 antigen alone results in a similar T cell response as immunization with a viral vector encoding gD. Treatment with the combination is more effective over time than immunization with gD alone. The HSV-2 is less likely to evade immune detection because the combined T cell response is stronger. The combination significantly reduces lesions in infected and prophylactically treated animals compared to non-immunized animals.

### II. Definitions

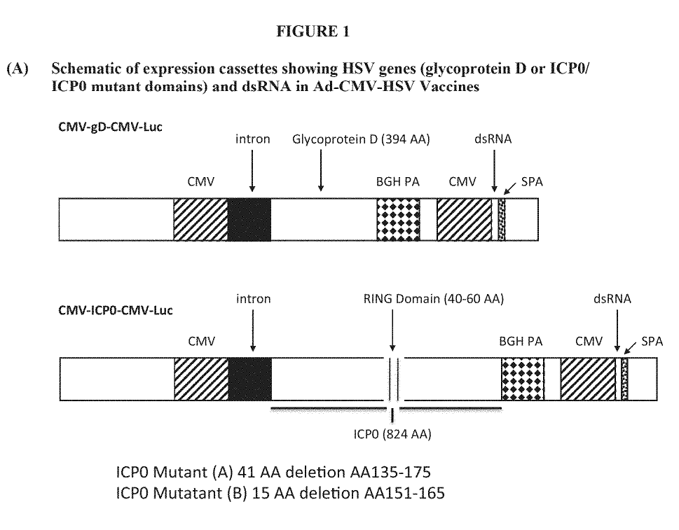

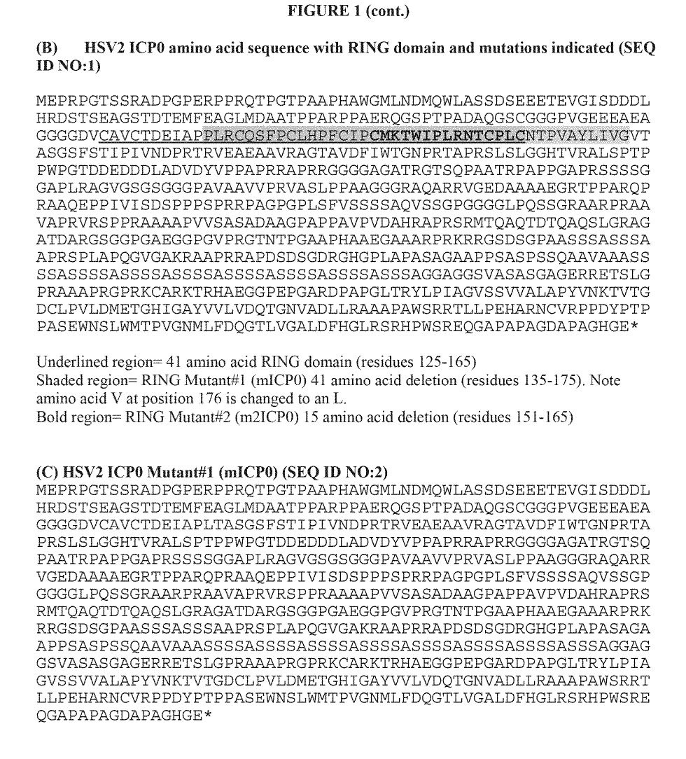

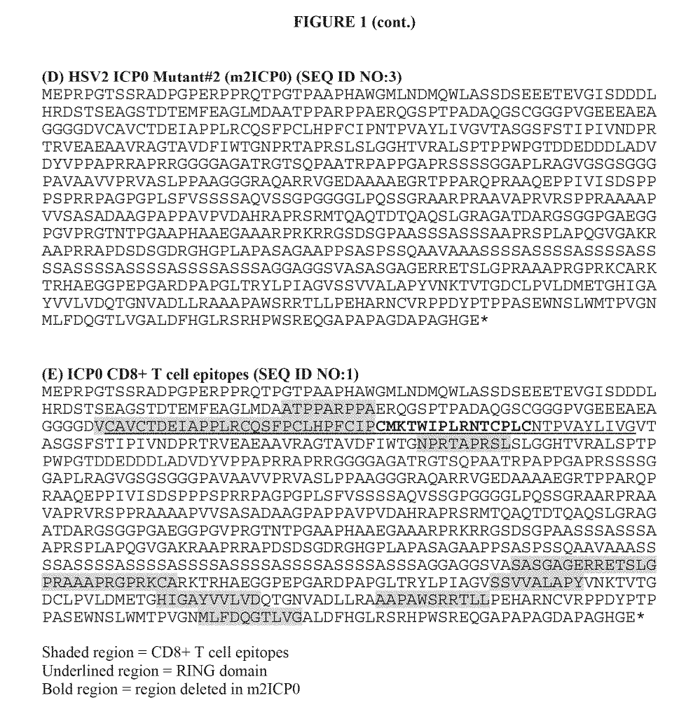

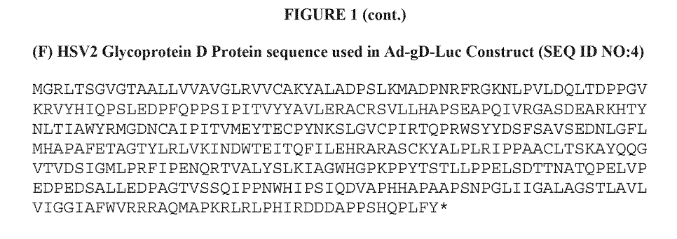

As used herein, the terms “ICP0 antigen,” “mutant ICP0 antigen,” “mutant ICP0 polypeptide,” and like terms refer to a polypeptide derived from the wild type HSV-2 ICP0 polypeptide that has been manipulated (e.g., using recombinant methods). In some embodiments, the mutation reduces the pro-viral function of ICP0, e.g., ubiquitin ligase activity, or gene expression and activation activities, while maintaining at least one T cell antigen (see. e.g., FIG. 1E). The terms “wild type” or “naturally occurring” are used to refer to the non-manipulated form of ICP0. The ICP0 antigen can represent a fragment of wild type ICP0 (SEQ ID NO:1), e.g., at least 20%, 30%, 40%, 50%, 60%, 70%, 80%, 85%, 90%, 95%, 25-50%, 50-75%, or 50-99% of the length of wild type ICP0. The ICP0 antigen can also be a mutated form of wild type ICP0 or a fragment thereof. For example, the ICP0 antigen can have at least 50, 60, 70, 75, 80, 85, 90, 95, 98, or 99% sequence identity with wild type ICP0 (or the fragment thereof). “Wild type” ICP0 includes polypeptides from various isolates of HSV-2. Examples include the sequence shown in FIG. 1B (SEQ ID NO: 1), and GenBank Accession Numbers: GU979901.1, GU979903.1, GU979902.1, GU979900.1, and GU979898.1. These all have a RING (Really Interesting New Gene) domain of 40-60 amino acids that can bind two zinc atoms, defined by the consensus sequence: C—X2-C—X(9-39)-C—X(1-3)-H—X(2-3)-(N/C/H)—X2-C—X(4-48)-C—X2-C(SEQ ID NO:5). In some embodiments, the ICP0 antigen has a mutation (insertion, substitution, and/or deletion) in the RING domain. In some embodiments, the ICP0 antigen has 1, 2, 3, 4, 5, 6, 7, or 8 of the conserved amino acids in the RING domain substituted or deleted. In some embodiments, the spacing of the conserved amino acids in the RING domain is disrupted e.g., by a deletion or addition. In some embodiments, the ICP0 antigen has reduced ubiquitin ligase activity compared to a wild type HSV-2 ICP0 polypeptide, e.g., less than 90%, 80%, 70%, 50%, 25%, 20%, 10%, 5%, 2%, or 1% wild type ubiquitin ligase activity. Examples of ICP0 antigens are mICP0 (SEQ ID NO:2) and m2ICP0 (SEQ ID NO:3).

A mutant sequence is one that is modified from the wild type or predominantly occurring sequence. The sequence can be polypeptide or polynucleotide sequence, and can refer to a single amino acid or nucleic acid. The mutation can be a substitution, insertion, or deletion of amino acids or nucleic acids. When multiple amino acids or nucleic acids are mutated, they can be consecutive or non-consecutive in the mutated sequence. Mutations can be naturally occurring or the result of manipulation, e.g., using recombinant techniques or irradiation. In the context of the present disclosure, unless otherwise indicated, the mutation is the result of non-naturally occurring manipulation.

The term “chimeric” or “recombinant” as used herein with reference, e.g., to a nucleic acid, protein, vector, or cell indicates that the nucleic acid, protein, vector, or cell has been modified by the introduction of a heterologous nucleic acid or protein or the alteration of a native nucleic acid or protein. Thus, for example, recombinant vectors include nucleic acid sequences that are not found within the native (non-chimeric or non-recombinant) form of the vector. A chimeric adenoviral expression vector refers to an adenoviral expression vector comprising a nucleic acid sequence encoding a heterologous polypeptide.

An “expression vector” is a nucleic acid construct, generated recombinantly or synthetically, with a series of specified nucleic acid elements that permit transcription of a particular nucleic acid in a host cell. The expression vector can be a plasmid, virus, or nucleic acid fragment. Typically, the expression vector includes a nucleic acid to be transcribed operably linked to a promoter.

The terms “promoter” and “expression control sequence” refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter can optionally includes distal enhancer or repressor elements. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “TLR-3 agonist” or “Toll-like receptor 3 agonist” as used herein refers to a compound that binds and stimulates the TLR-3. TLR-3 agonists include double-stranded RNA, virally derived dsRNA, several chemically synthesized analogs to double-stranded RNA including polyinosine-polycytidylic acid (poly I:C) -polyadenylic-polyuridylic acid (poly A:U) and poly I:poly C, and antibodies (or cross-linking of antibodies) to TLR-3 that lead to IFN-beta production (Matsumoto, M, el al. Biochem Biophys Res Commun 24:1364 (2002), de Bouteiller, et al, J Biol Chem 18:38133-45 (2005)). TLR-3 agonists also include expressed dsRNA.

An “antigen” refers to a protein or part of a polypeptide chain that can be recognized by T cell receptors and/or antibodies. Typically, antigens are derived from bacterial, viral, or fungal proteins. The term “epitope” refers to the portion of the antigen that is recognized by the T cell receptor or antibody. Typically, the term antigen is interpreted to be broader than the term epitope. For example, a T cell receptor or antibody might be specific for a given antigen (e.g., protein X), and recognize or bind to only a few amino acids of protein X, the epitope. A “T cell epitope” is recognized or bound by a T cell receptor.

An “immunogenically effective dose or amount” of the presently disclosed compositions is an amount that elicits or modulates an immune response specific for the desired polypeptide, e.g., the ICP0 antigen or other HSV-2 antigen. Immune responses include humoral immune responses and cell-mediated immune responses. An immunogenic composition can be used therapeutically or prophylactically to treat or prevent HSV-2 infection and outbreak at any stage.

“Humoral immune responses” are mediated by cell free components of the blood, i.e., plasma or serum; transfer of the serum or plasma from one individual to another transfers immunity.

“Cell mediated immune responses” are mediated by antigen specific lymphocytes; transfer of the antigen specific lymphocytes from one individual to another transfers immunity.

A “therapeutic dose” or “therapeutically effective amount” or “effective amount” of a viral vector or a composition comprising a viral vector is an amount of the vector or composition comprising the vector which prevents, alleviates, abates, or reduces the severity of symptoms of HSV.

An antibody or immunoglobulin a polypeptide encoded by an immunoglobulin gene or a fragment thereof (e.g., Fab or F(ab)₂) that specifically bind and recognizes an antigen. Immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, igD and IgE, respectively.

T cells are lymphocytes that express a specific receptor (T cell receptor) encoded by a family of genes. T cell receptor genes include alpha, beta, delta, and gamma loci, and the T cell receptors typically (but not universally) recognize a combination of MHC plus a short peptide.

An adaptive immune response involves T cell and/or antibody recognition of antigen.

An “adjuvant” is a non-specific immune response enhancer.

The terms “nucleic acid” and “polynucleotide” are used interchangeably herein to refer to deoxyribonucleotide or ribonucleotide polymers in either single- or double-stranded form. A “nucleotide” typically refers to the monomer. The terms encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., a γ carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” apply to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine I, Lysine (K);
5) Isoleucine (I), Leucine (L), Methionine (M). Valine (V);
6) Phenylalanine (F), Tyrosine (Y), Tryptophan (W);
7) Serine (S), Threonine (T); and
8) Cysteine (C), Methionine (M)
(see. e.g., Creighton, Proteins (1984)).

The terms “identical” or percent “identity,” in the context of two or more nucleic acids (e.g., a dsRNA sequence) or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%/o, or more identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 10 to about 100, about 20 to about 75, about 30 to about 50 amino acids or nucleotides in length. A suitable algorithm for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (at the website available at ncbi.nlm.nih.gov).

### III. Antigens and Immunogenic Compositions

As indicated above, ICP0 is an immediate early gene in HSV with myriad roles in activating viral replication and reducing host defense (e.g., NFκB and IFN activation). ICP0 is a promiscuous activator of both viral and cellular promoters and can function synergistically with another immediate-early protein, ICP4. During viral infection, ICP0 can associate with a number of cellular proteins, including elongation factor 1δ, cyclin D3, kinetochore protein CENP-C, ubiquitin-specific protease 7 (USP7, also known as HAUSP), and PML, the prototypic member of nuclear domains known as ND10, PML bodies, or PODs. The RING domain has E3 Ubiquitin ligase activity, which is involved in the ubiquination of host cell proteins, thereby targeting them for destruction. Mutations in ICP0, including those in the RING domain, can significantly reduce the virulence of HSV and also reduce the activation of latent HSV. In some embodiments, the ICP0 antigen (mutant ICP) is mutated in the RING domain, e.g., with 1-60 of the amino acids in the RING domain replaced and/or deleted (e.g., 2-10, 5-15, 12-25, 20-30, 25-35, 30-40, 35-50 or 40-60 amino acid substitutions or deletions). In some embodiments, the ICP0 antigen includes at least 1, 2, 3, 4, 5, 6, 7, or 8 CD8+ T cell epitopes, e.g., epitopes found in wild type ICP0.

Ubiquitin ligase activity can be determined according to known methods, e.g., as described in Yasunaga et al. (2013) Mol. Cell. Biol. 33:644. Kits are commercially available, e.g., E3LITE Customizable Ubiquitin Ligase Kit (Lifesensors), which can be used to detect E3 ubiquitin ligase activity in a given protein sample.

Additional HSV antigens that can be used in combination with the presently described ICP0 antigens include viral capsid, envelope, or tegument proteins. Examples include UL4, UL6, UL8, UL9, UL14, UL18, UL19, UL29, UL35, UL38, glycoproteins B, C, D, E, G, I, and J. As with the ICP0 antigen, a fragment or modified (mutant) form of the other HSV-2 antigen can be used. For example, an immunogenic fragment or mutant of the selected HSV-2 antigen can be designed such that it does not have significant activity to neutralize host immune response or promote HSV replication or activation. The immunogenic fragment or mutant can then be used in combination with the ICP0 antigen described herein.

### IV. Recombinant Methods

The nucleic acids encoding immunogenic polypeptides (e.g., ICP0 antigen or other HSV-2 antigen), are typically produced by recombinant DNA methods (see, e.g., Ausubel, et al. ed. (2001) Current Protocols in Molecular Biology). For example, the DNA sequence encoding the immunogenic polypeptide can be assembled from cDNA fragments and short oligonucleotide linkers, or from a series of oligonucleotides, or amplified from cDNA using appropriate primers to provide a synthetic gene which is capable of being inserted into a recombinant expression vector (i.e., a plasmid vector or a viral vector) and expressed in a recombinant transcriptional unit. Once the nucleic acid encoding an immunogenic polypeptide is produced, it may be inserted into a recombinant expression vector that is suitable for in vivo or ex-vivo expression.

Recombinant expression vectors contain a DNA sequence encoding an immunogenic polypeptide operably linked to suitable transcriptional or translational regulatory elements derived from mammalian or viral genes. Such regulatory elements include a transcriptional promoter, an optional operator sequence to control transcription, a sequence encoding suitable mRNA ribosomal binding sites, and sequences which control the termination of transcription and translation. An origin of replication and a selectable marker to facilitate recognition of transformants may additionally be incorporated. The genes utilized in the recombinant expression vectors can be divided between more than one viral vector such that the gene products are on two different vectors, and the vectors are used for co-transduction to provide all the gene products in trans. There may be reasons to divide up the gene products such as size limitations for insertions.

The presently disclosed immunogenic compositions can be delivered on a heterologous viral vector. In some embodiments, the viral vector is an adenoviral vector. The adenoviral vector can be human isolated Ad species such as Ad5, Ad4, Ad7, Ad26, Ad40, Ad41, or non-human primate derived adenovirus such as chimpanzee derived species of Ad. Other vectors that can be used include lentiviral, VSV, Sindbis, BEE, and AAV. In some embodiments, the vector comprises a first promoter operably linked to a nucleic acid encoding an ICP0 antigen. In some embodiments, the vector further comprises a second promoter operably linked to a nucleic acid encoding a TLR3 agonist, e.g., dsRNA. In some embodiments, the vector further comprises a second promoter operably linked to a nucleic acid encoding another HSV-2 antigen. In some embodiments, the vector comprises all three expression cassettes. The first and second (and optionally third) promoters can be the same or different. In some embodiments, the first and second (and optionally third) promoters are independently selected from the beta actin promoter and the CMV promoter.

In some embodiments, the heterologous vector is an adenoviral vector comprising the adenoviral genome (minus the E1 and E3 genes) and a nucleic acid encoding a gene that activates IRF-3 and other signaling molecules downstream of TLR-3. The chimeric vector can be administered to a cell that expresses the adenoviral E1 gene such that recombinant adenovirus (rAd) is produced by the cell. This rAd can be harvested and is capable of a single round of infection that will deliver the transgenic composition to another cell within a mammal in order to elicit immune responses to the immunogenic polypeptide.

In some embodiments, the adenoviral vector is adenovirus 5, including, for example, Ad5 with deletions of the E1/E3 regions and Ad5 with a deletion of the E4 region. Other suitable adenoviral vectors include strains 2, orally tested strains 4 and 7, enteric adenoviruses 40 and 41, and other strains (e.g. Ad34) that are sufficient for delivering an antigen and eliciting an adaptive immune response to the transgene antigen (Lubeck et al., Proc Natl Acad Sci USA, 86(17), 6763-6767 (1989); Shen el al., J Virol, 75(9), 4297-4307 (2001); Bailey et al., Virology, 202(2), 695-706 (1994)). In some embodiments, the adenoviral vector is a live, replication incompetent adenoviral vector (such as E1 and E3 deleted rAd5), live and attenuated adenoviral vector (such as the E1B55K deletion viruses), or a live adenoviral vector with wild-type replication.

The transcriptional and translational control sequences in expression vectors to be used in transforming vertebrate cells in vivo may be provided by viral sources. For example, commonly used promoters and enhancers are derived, e.g., from beta actin, adenovirus, simian virus (SV40), and human cytomegalovirus (CMV). For example, vectors allowing expression of proteins under the direction of the CMV promoter, SV40 early promoter, SV40 later promoter, metallothionein promoter, murine mammary tumor virus promoter. Rous sarcoma virus promoter, transducer promoter, or other promoters shown effective for expression in mammalian cells are suitable. Further viral promoter, control and/or signal sequences can be used, provided such control sequences are compatible with the host cell chosen.

### V. Pharmaceutical Compositions

Provided herein are pharmaceutical compositions for HSV-2 vaccination of uninfected and infected individuals. The pharmaceutical composition can be formulated for oral or mucosal delivery as described below. The pharmaceutical composition can also be formulated for injection (e.g., intravenous, intramuscular, intraperitoneal, subcutaneous, etc.). In some embodiments, the pharmaceutical composition comprises an ICP0 antigen (mutant ICP0) polypeptide (e.g., mICP0 or m2ICP0), optionally in combination with dsRNA (or a dsRNA mimetic), and optionally another HSV-2 antigen (e.g., a capsid protein such as gD or gB). In some embodiments, the pharmaceutical composition comprises an expression cassette encoding the ICP0 antigen, e.g., in a heterologous expression vector. In some embodiment the composition comprises a viral vector encoding an ICP0 antigen, and optionally dsRNA, and optionally another HSV-2 antigen (e.g., a capsid protein such as gD or gB). In some embodiments, the polynucleotide encoding the dsRNA and/or HSV-2 antigen is delivered on a separate viral vector but also included in the same pharmaceutical composition. In some embodiments, the separate viral vector is delivered in a separate pharmaceutical composition. In some embodiments, the pharmaceutical composition further includes dsRNA or a dsRNA mimetic, i.e., not encoded on a viral vector. In some embodiments, the pharmaceutical composition further includes an HSV-2 antigen, e.g., a capsid protein, not encoded on a viral vector.

Pharmaceutical compositions comprising the compositions described herein can contain other compounds, which may be biologically active or inactive. Pharmaceutical compositions can be composed to protect against stomach degradation such that the administered composition (e.g., viral vector) reaches the desired location (e.g., ileum). See, e.g., U.S. Ser. No. 61/942,386. For the oral environment, several of these are available including the Eudragit and the TimeClock release systems as well as other methods specifically designed for adenovirus (Lubeck et al., Proc Natl Acad Sci USA, 86(17), 6763-6767 (1989); Chourasia and Jain, J Pharm Pharm Sci, 6(1), 33-66 (2003)). There are also several methods already described for microencapsulation of DNA and drugs for oral delivery (see, e.g., U.S. Patent Publication No. 2004043952). In some embodiments, the Eudragit system is used to deliver the viral vector to the lower small intestine, or to another location of the small intestine.

The presently described compositions can be delivered using any delivery system known to those of ordinary skill in the art. Numerous gene delivery techniques are well known in the art, such as those described by Rolland (1998) Crit. Rev. Therap. Drug Carrier Systems 15:143-198, and references cited therein.

The presently described immunogenic compositions can contain pharmaceutically acceptable salts. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts). Some particular examples of salts include phosphate buffered saline and saline for injection.

Any suitable carrier known to those of ordinary skill in the art can be employed in the pharmaceutical compositions described herein. Suitable carriers include, for example, water, saline, alcohol, a fat, a wax, a buffer, a solid carrier, such as mannitol, lactose, starch, magnesium stearate, sodium saccharine, talcum, cellulose, glucose, sucrose, and magnesium carbonate, or biodegradable microspheres (e.g., polylactate polyglycolate). Suitable biodegradable microspheres are disclosed, for example, in U.S. Pat. Nos. 4,897,268; 5,075,109; 5,928,647; 5,811,128; 5,820,883. The immunogenic polypeptide and/or carrier virus can be encapsulated within the biodegradable microsphere or associated with the surface of the microsphere.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. The presently described compositions can also be lyophilized, or encapsulated within liposomes using well known technology.

In some embodiments, the presently disclosed compositions further comprise an adjuvant such as a TLR-3 agonist (e.g., dsRNA or a mimetic thereof such as poly I:C or poly A:U). A TLR-3 agonist is used to stimulate immune recognition of an antigen of interest. TLR-3 agonists include, for example, short hairpin RNA, virally derived RNA, short segments of RNA that can form double-strands or short hairpin RNA, and short interfering RNA (siRNA). The TLR-3 agonist can be virally derived dsRNA, such as for example, a dsRNA derived from a Sindbis virus or dsRNA viral intermediates (Alexopoulou et al. Nature 413:732-8 (2001)). In some embodiments, the TLR-3 agonist is a short hairpin RNA. Short hairpin RNA sequences typically comprise two complementary sequences joined by a linker sequence. The particular linker sequence is not critical. Any linker sequence can be used so long as it does not interfere with the binding of the two complementary sequences to form a dsRNA.

Other suitable adjuvants include, for example, the lipids and non-lipid compounds, cholera toxin (CT), CT subunit B, CT derivative CTK63, E. coli heat labile enterotoxin (LT), LT derivative LTK63, Al(OH)₃, and polyionic organic acids as described in e.g., WO 04/020592, Anderson and Crowle, Infect. Immun. 31(1):413-418 (1981), Roterman et al., J. Physiol. Pharmacol., 44(3):213-32 (1993), Arora and Crowle. J. Reticuloendothel. 24(3):271-86 (1978), and Crowle and May, Infect. Immun. 38(3):932-7 (1982)). Suitable polyionic organic acids include for example, 6,6′-[3,3′-demithyl[1,1′-biphenyl]-4,4′-diyl]bis(azo)bis[4-amino-5-hydroxy-1,3-naphthalene-disulfonic acid] (Evans Blue) and 3,3′-[1,1′biphenyl]-4,4′-diylbis(azo)bis[4-amino-1-naphthalenesulfonic acid] (Congo Red).

Other suitable adjuvants include topical immunomodulators such as, members of the imidazoquinoline family such as, for example, imiquimod and resiquimod (see, e.g., Hengge et al., Lancet Infect. Dis. 1(3):189-98 (2001).

Additional suitable adjuvants are commercially available as, for example, additional alum-based adjuvants (e.g., Alhydrogel, Rehydragel, aluminum phosphate, Algammulin); oil based adjuvants (Freund's Incomplete Adjuvant and Complete Adjuvant (Difco Laboratories, Detroit. Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A and Quil A. Cytokines, such as GM-CSF or interleukin-2, -7, or -12, are also suitable adjuvants. Hemocyanins (e.g., keyhole limpet hemocyanin) and hemoerythrins may also be used in the invention. Polysaccharide adjuvants such as, for example, chitin, chitosan, and deacetylated chitin are also suitable as adjuvants. Other suitable adjuvants include muramyl dipeptide (MDP, N acetylmuramyl L alanyl D isoglutamine) bacterial peptidoglycans and their derivatives (e.g., threonyl-MDP, and MTPPE). BCG and BCG cell wall skeleton (CWS) may also be used as adjuvants in the invention, with or without trehalose dimycolate. Trehalose dimycolate may be used itself (see, e.g., U.S. Pat. No. 4,579,945). Detoxified endotoxins are also useful as adjuvants alone or in combination with other adjuvants (see, e.g., U.S. Pat. Nos. 4,866,034; 4,435,386; 4,505,899; 4,436,727; 4,436,728; 4,505,900; and 4,520,019. The saponins QS21, QS17, QS7 are also useful as adjuvants (see, e.g., U.S. Pat. No. 5,057,540; EP 0362 279; WO 96/33739; and WO 96/11711). Other suitable adjuvants include Montanide ISA 720 (Seppic, France), SAF (Chiron, Calif., United States), ISCOMS (CSL), MF-59 (Chiron), the SBAS series of adjuvants (e.g., SBAS-2, SBAS-4 or SBAS-6 or variants thereof, available from SmithKline Beecham, Rixensart, Belgium), Detox (Corixa, Hamilton. Mont.), and RC-529 (Corixa, Hamilton, Mont.).

Within the pharmaceutical compositions provided herein, the adjuvant composition can be designed to induce, e.g., an immune response predominantly of the Th1 or Th2 type. High levels of Th1-type cytokines (e.g., IFN-gamma, TNF-alpha, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Th1- and Th2-type responses will typically be elicited following oral or mucosal delivery of a composition as provided herein.

The compositions described herein may be administered as part of a sustained release formulation (i.e., a formulation such as a capsule or sponge that effects a slow release of compound following administration). Such formulations may generally be prepared using well known technology (see, e.g., Coombes el al. (1996) Vaccine 14:1429-1438). Sustained-release formulations may contain a polypeptide, polynucleotide or antibody dispersed in a carrier matrix and/or contained within a reservoir surrounded by a rate controlling membrane.

Carriers for use within such formulations are biocompatible, and can provide a relatively constant level of active component release. Such carriers include microparticles of poly(lactide-co-glycolide), as well as polyacrylate, latex, starch, cellulose and dextran. Other delayed-release carriers include supramolecular biovectors, which comprise a non-liquid hydrophilic core (e.g., a cross-linked polysaccharide or oligosaccharide) and, optionally, an external layer comprising an amphiphilic compound (see, e.g., WO 94/20078; WO 94/23701; and WO 96/06638). The amount of active compound contained within a sustained release formulation depends upon the site of implantation, the rate and expected duration of release and the nature of the condition to be treated or prevented.

The pharmaceutical compositions can be packaged in unit-dose or multi-dose containers, such as sealed ampoules or vials. Such containers can be hermetically sealed to preserve integrity or sterility of the formulation until use. Formulations can be stored as suspensions, solutions, or emulsions in oily or aqueous vehicles, in a lyophilized condition (e.g., for addition of sterile liquid carrier before use), or in an oral delivery formulation, e.g. capsule, tablet, or pill.

### VI. Therapeutic Methods

Administration of the presently disclosed compositions can be by any non-parenteral route (e.g., vaginally, orally, intranasally, or otherwise mucosally via, e.g., lungs, salivary glands, nasal cavities, small intestine, colon, rectum, tonsils, or Peyer's patches), or any parenteral route (e.g., intramuscular, subcutaneous, intraperitoneal, intravenous, etc.). The composition can be administered alone or with an adjuvant. In some embodiments, the adjuvant(s) is encoded by a nucleic acid sequence (e.g., a nucleic acid encoding dsRNA), e.g., on the same vector or on a separate vector as the ICP0 antigen. In some embodiments, the adjuvant is administered at the same time as the composition. In some embodiments, the adjuvant is administered after the composition, e.g., 1, 2, 6, 12, 18, 24, 36, 48, 60, or 72 hours after administration of the composition.

In addition the presently disclosed compositions can be administered in combination with other immunogenic compositions, as described above. The viral vector encoding the ICP0 antigen can be administered in combination with a viral vector encoding another HSV-2 antigen, such as gD or gB. Administration can be concurrent (e.g., in a single pharmaceutical composition) or sequential, e.g., 1, 2, 6, 12, 18, 24, 36, 48, 60, or 72 hours apart. In some embodiments, one or the other or both viral vectors also encode dsRNA. In some embodiments, the viral vector encoding the ICP0 antigen is administered with another HSV-2 antigen, e.g., a protein antigen. Again, administration can be concurrent or sequential.

The presently disclosed compositions can be administered prophylactically, to an individual that does not have detectable HSV, has not displayed symptoms of HSV infection, or one that is at risk of infection. The presently disclosed compositions can also be administered to reduce severity of HSV symptoms in an individual that is already infected, and reduce the likelihood of the individual spreading the virus (e.g., by reducing viral shedding).

Frequency of administration of the prophylactic or therapeutic compositions described herein, as well as dosage, will vary from individual to individual, and can be readily established using standard techniques. Between 1 and 10 doses may be administered over a 52 week period. In some embodiments, the presently disclosed composition is administered upon early indication of an outbreak. In some embodiments, administration is once/year. In some embodiments, 3 doses are administered, at intervals of 1 month, or 2-3 doses are administered every 2-3 months. Booster vaccinations can be given periodically thereafter. Alternate protocols may be appropriate for individual patients and particular diseases and disorders. A suitable dose is an amount of the composition that, when administered as described above, is capable of promoting an anti-viral immune response, and is at least 10-50% above the basal (i.e., untreated) level. Such response can be monitored by measuring vaccine-dependent generation or activation of cytolytic CD8 T cells capable of killing virally infected cells, e.g., as determined in vitro. Such vaccines should also be capable of causing an immune response that leads to an improved clinical outcome (e.g., less frequent outbreaks, or complete or partial remission) in vaccinated as compared to non-vaccinated individuals. Those of skill in the art will appreciate that the dose size may be adjusted based on the particular patient. For oral administration, the presently disclosed compositions can conveniently be formulated in a coated tablet, pill, or capsule. For vaginal or other mucosal administration, gel, ointment, or suppository can be used.

An appropriate dosage and treatment regimen provides the presently disclosed compositions in an amount sufficient to provide therapeutic and/or prophylactic benefit. Such a response can be monitored by establishing an improved clinical outcome (e.g., less frequent outbreaks, prevention of appearance of symptoms, complete or partial, reduced rate of spreading the infection) in treated individuals as compared to non-treated individuals. Immune responses to the presently disclosed compositions can be evaluated using standard proliferation, cytotoxicity or cytokine assays, which may be performed using samples obtained from a patient before and after treatment.

An immune response to a given antigen can be detected using any means know in the art including, for example, detecting specific activation of CD4⁺ or CD8⁺ T cells or by detecting the presence of antibodies that specifically bind to the polypeptide.

Specific activation of CD4⁺ or CD8⁺ T cells associated with a mucosal, humoral, or cell-mediated immune response can be detected in a variety of ways. Methods for detecting specific T cell activation include, but are not limited to, detecting the proliferation of T cells, the production of cytokines (e.g., lymphokines), or the generation of cytolytic activity (i.e., generation of cytotoxic T cells specific for the immunogenic polypeptide). For CD4⁺ T cells, specific T cell activation is indicated by proliferation of T cells. For CD8⁺ T cells, specific T cell activation is indicated by the generation of cytolytic activity, e.g., detectable using ⁵¹Cr release assays (see, e.g., Brossart and Bevan, Blood 90(4): 1594-1599 (1997) and Lenz et al., J. Exp. Med. 192(8):1135-1142 (2000)).

Detection of the proliferation of T cells may be accomplished by a variety of known techniques. For example, T cell proliferation can be detected by measuring the rate of DNA synthesis in T cells (e.g., isolated CD8 T cells). A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyltetrazolium. Alternatively, synthesis of lymphokines (e.g., interferon-gamma) can be measured as indication of T cell response, or the relative number of T cells that can respond to the immunogenic polypeptide (e.g., ICP0 or other HSV-2 antigen) can be quantified.

Antibody immune responses (humoral immune responses or B cell responses), including mucosal antibody responses can be detected using immunoassays known in the art (see, e.g., Tucker el al., Mol Therapy, 8, 392-399 (2003); Tucker el al., Vaccine, 22, 2500-2504 (2004)). Suitable immunoassays include the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al. (1980) J. Biol. Chem. 255:4980-4983); enzyme-linked immunosorbent assays (ELISA) as described, for example, by Raines et al. (1982) J. Biol. Chem. 257:5154-5160; immunocytochemical techniques, including the use of fluorochromes (Brooks et al. (1980) Clin. Exp. Immunol. 39:477); and neutralization of activity (Bowen-Pope el al. (1984) Proc. Natl. Acad. Sci. USA. 81:2396-2400). In addition to the immunoassays described above, a number of other commercially available immunoassays can be used, e.g., to detect antibodies specific for HSV-2 antigens, such as ICP0 or an HSV-2 capsid protein (e.g, gD or gB).

### VII. Examples

### A. Example 1: Generation of Adenoviral Vectors Expressing ICP0 Antigens

We generated Ad vectors expressing wild-type ICP0 and mutant forms of the ICP0 which still retain previously identified T cell epitopes. See FIG. 1. The adenoviral vector is Ad5, with E1/E3 deleted. The Ad5 construct is suitable for therapeutic delivery of recombinant antigens as described, e.g., in U.S. Pat. Nos. 7,879,602 and 8,222,224.

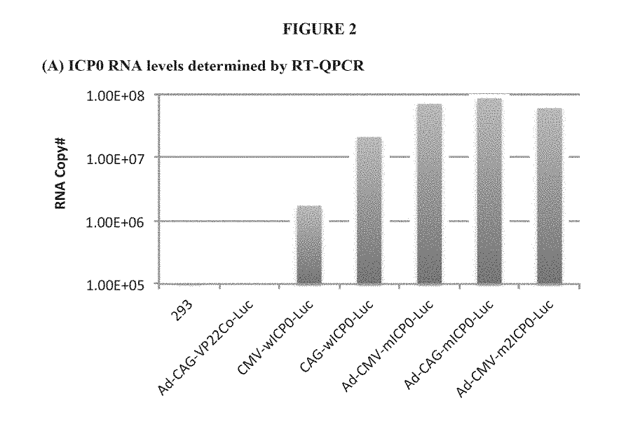

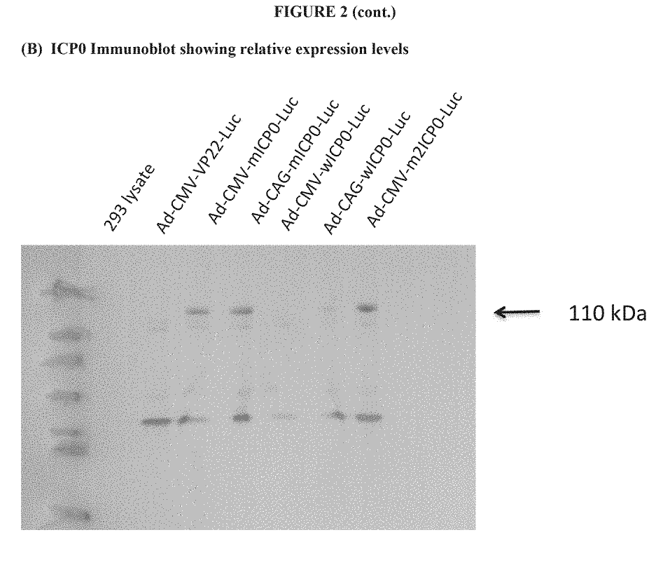

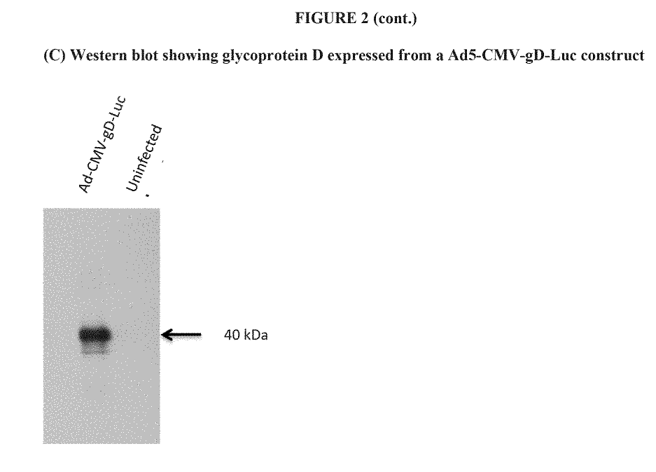

FIG. 1A provides a schematic of HSV transgene constructs. The first vaccine construct expresses a 394 amino acid glycoprotein D protein under the control of a CMV promoter with a β-globin intron and a bovine growth hormone polyA. The sequence from the glycoprotein D is based on Genbank accession number NP_044536 (FIG. 1F; SEQ ID NO:4). The second construct expresses wild type (FIG. 1B; SEQ ID NO:1) or mutant forms of ICP0 (FIGS. 1C and 1D; SEQ ID NOs:2 and 3). The CMV promoter is shown in FIG. 1A, but a CAG promoter could be used to boost ICP0 expression (see FIGS. 2A and 2B). Two ICP0 mutants were made. They were based on the original wild type ICP0 sequence referenced in Genbank accession number NP_044469 (SEQ ID NO:1). RING Mutant#1 (mICP0) consists of a 41 AA large deletion between AA135-175 in the RING domain. In addition, the Valine at 176 was mutated to a Leucine (FIG. 1C). Ring Mutant#2 (m2ICP0) has a smaller deletion of only 15 AA from position 151-165 (FIG. 1D). m2ICP0 was constructed to reduce biological activity of ICP0, but to preserve two overlapping T cell epitopes between amino acids 123-150 of ICP0 (see FIG. 1E).

Expression was evaluated in vitro following infection of HEK293 cells. HEK293 cells were infected at an MOI (multiplicity of infection) of 1, and 48 h later the cells were harvested and RNA extracted. cDNA was made by reverse transcription and copy number determined by QPCR. RT minus controls and a GAPDH standard were included. FIG. 2A shows the RNA copy number amplified from 1 ul of cDNA generated using 1 ug of total RNA. The minus RT controls had low or negligible RNA copies detected and the samples were normalized with GAPDH. The CMV and CAG ICP0 wild type constructs had lower levels of RNA compared to CMV mutant 1 (mICP0) and CAG mutant 1, and CMV-mutant 2 (m2ICP0).

FIG. 2B shows a Western Blot from lysates of infected cells with the various ICP0 constructs. Protein levels also appeared to follow the same trend as the RNA levels, with the mutants having higher levels of ICP0 expression than the wild type constructs. No protein was detected for the CMV wild-type ICP0 and a weak signal was detected for the CAG wild-type ICP0. Expression of gD was also confirmed for the Ad-CMV-gD-Luc in a similar fashion as described for ICP0 (FIG. 2C).

### B. Example 2: HSV-2 Vaccination in Murine Model

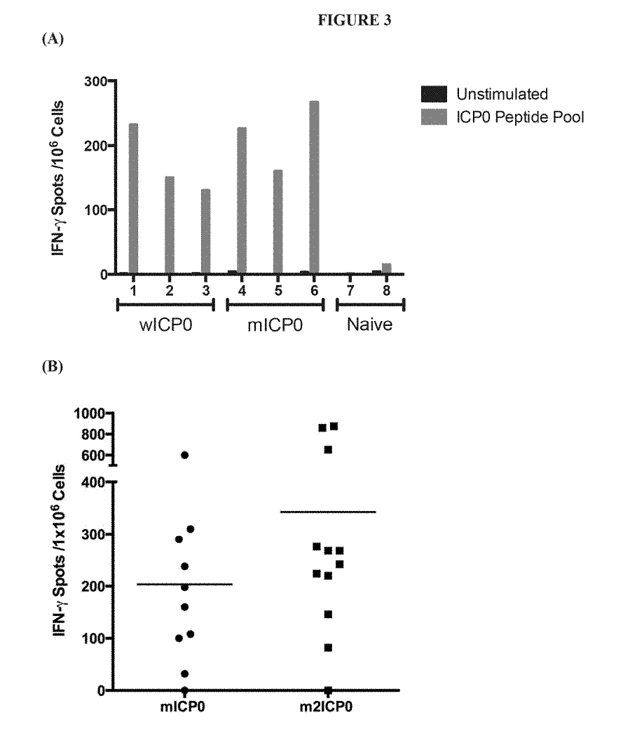

In order to demonstrate the ability of mutant forms of ICP0 to elicit a T cell response in a gene-based approach, rAd vectors expressing the two mutant forms of ICP0 were tested (FIG. 3). First, mice were immunized intramuscularly with 1e8 IU of vector per mouse with either the wildtype construct (wICP0) or a mutant construct (mICP0). The ability of each construct to elicit T cell responses to a full-length ICP0 peptide library was examined (FIG. 3A). The mutant construct (mICP0) elicited slightly more IFN-γ spot forming cells (215 SFC) than the wildtype construct (wICP0) (170 SFC) per 1e6 cells (FIG. 3A).

The second mutant ICP0 (m2ICP0) was also tested, and its ability to elicit T cell responses compared to the first mutant mICP0. Before vaccination mice were injected with deprovera to thin the epithelial lining of the vagina (see. e.g., Farley, N, et al, (2010) Antiviral Res, 86:188). Each construct was administered by intravaginal vaccination of 1e8 IU of vector (FIG. 3B). After vaccination, m2ICP0 generated a slightly greater number of IFN spot forming cells (275 SFC) compared to mICP0 (190 SFC) (FIG. 3B). The data strongly indicate that both mutant forms of ICP0 produce T cell responses greater than the wildtype construct (FIG. 3).

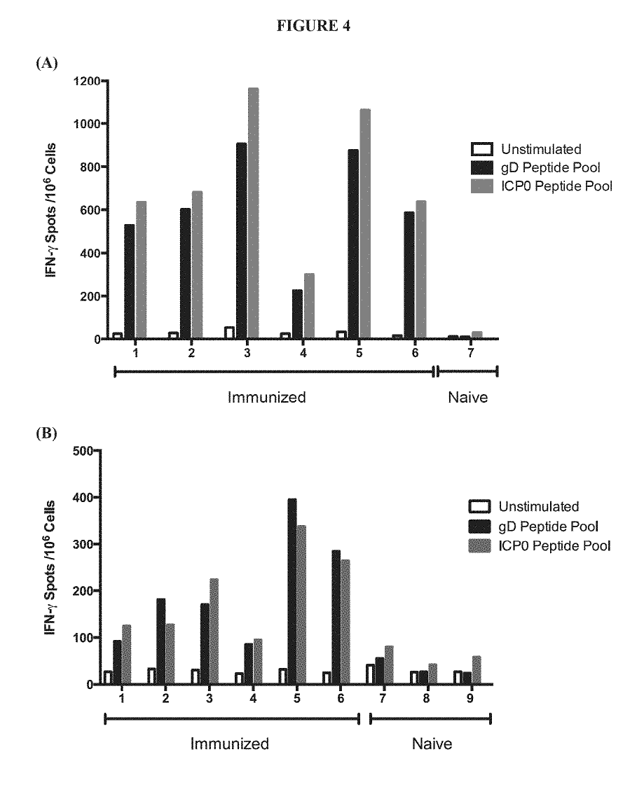

We next investigated the immune response to both the gD and mICP0 constructs described in Example 1. Both constructs were simultaneously administered intravaginally at a dose of 1e8 IU each. FIG. 4 shows immune responses in both draining iliac lymph nodes (ILN) and spleens measured by IFN-γ ELIspot after immunization with both gD peptide library pool (black bars) and ICP0 peptide library pool (grey bars). Similar responses to both gD and ICP0 (mean=506 SFC and 616 SFC respectively) in the ILNs, and gD and ICP0 (mean=117 SFC and 121 SFC respectively) in the spleen were observed. The results indicate that there is no disadvantage to the immune response when two vectors expressing different antigens are delivered together.

The ability to protect against HSV-2 or to treat HSV-2 infection would benefit greatly from vaginal homing of antigen specific T cells. Given that vaginal delivery would likely lead to the greatest recruitment of T cells, this route of vaccination was tested first. Mice were vaccinated intravaginally with rAd-mICP0-dsRNA. Homing of T cells was tested by direct digestion of the genital tract and isolation of resident T cells.

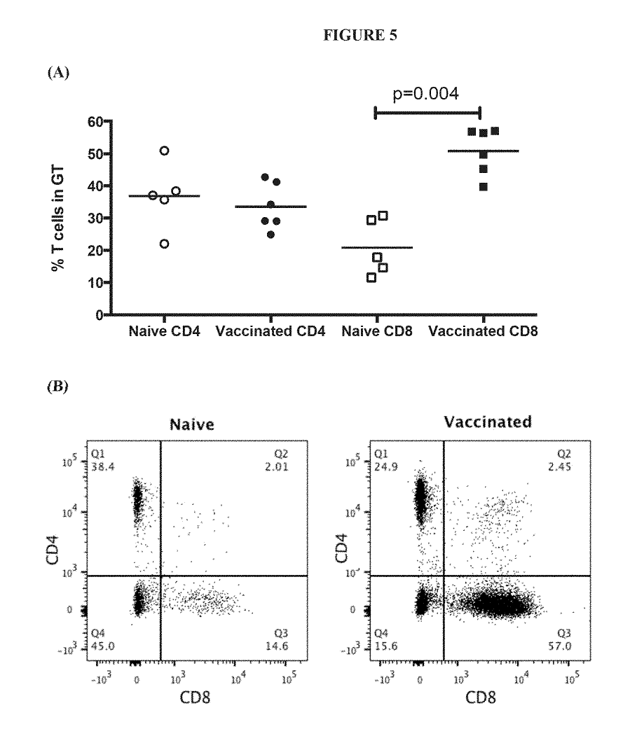

Seven days following the third vaccination with rAd-mICP0-dsRNA, genital tracts were isolated and the tissue enzymatically digested. Mononuclear cells were separated from epithelial cells and quantiated by flow cytometry using fluorescent antibodies to CD4 and CD8 T cell antigens. Significantly more CD8 positive T cells were found in immunized mice compared to naïve mice (p=0.004) (FIG. 5). The percentages of CD4 and CD8 positive T cells isolated from individual mice is depicted in FIG. 5A and a representative flow cytometry plot of a naïve and vaccinated mouse is shown in FIG. 5B.

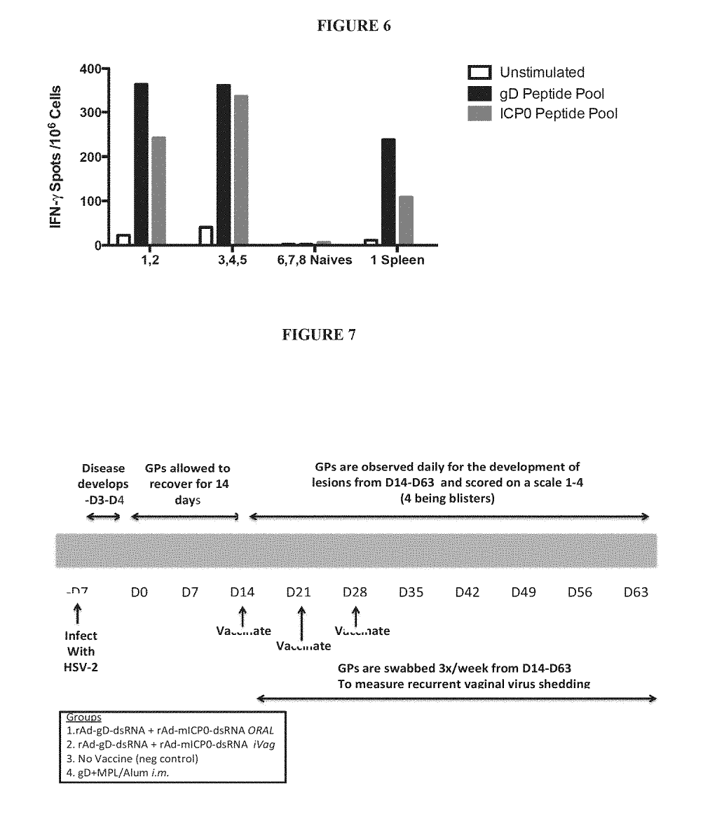

To ensure that the T cells homing to the genital tract were specifically active against the vaccine antigens (gD and mICP0), an ELIspot was performed using the cells isolated from the genital tract after enzymatic digestion and density gradient separation. Either 2 or 3 mice were pooled in order to stimulate the same number and concentration of cells with both the gD (black bars) and the mICP0 (grey bars) peptide pools. Similar T cell responses were found after gD and mICP0 stimulation in the ILNs (mean=332 SFC, and 258 SFC respectively) (FIG. 6).

### C. Example 3: HSV-2 Vaccination in a Guinea Pig Model

Guinea pigs are the preferred model for HSV-2 genital infection, and the ability of the rAd-mICP0-dsRNA to elicit therapeutic effects was tested following an initial experiment with rAd-gD-dsRNA. In the model (outlined in FIG. 7), guinea pigs are infected intravaginally with HSV-2 on day −7 and the disease develops for several days. Animals are allowed to recover for 14 days before immunizing, once a week for 3 weeks. Guinea pigs are monitored each day for lesion development and the cumulative daily average scores for each group are calculated. Guinea pigs were scored daily 0-4: 0=negative; 1=slight erythema (redness) or healing vesicles; 2=moderate erythema with swelling; 3=severe erythema with swelling and small vesicles; 4=severe erythema with swelling and large vesicles.

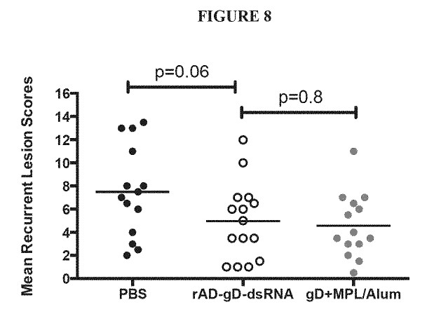

In the initial experiment, vaginal delivery of rAd-gD-dsRNA was tested for the ability to induce protective immune responses compared to gD protein +MPL/Alum and a negative control (unimmunized guinea pigs). Vaginal delivery of rAd-gD-dsRNA resulted in a decrease in the cumulative daily lesion score (p=0.06), similar to the gD protein +MPL/Alum (positive control) (p=0.8) (FIG. 8).

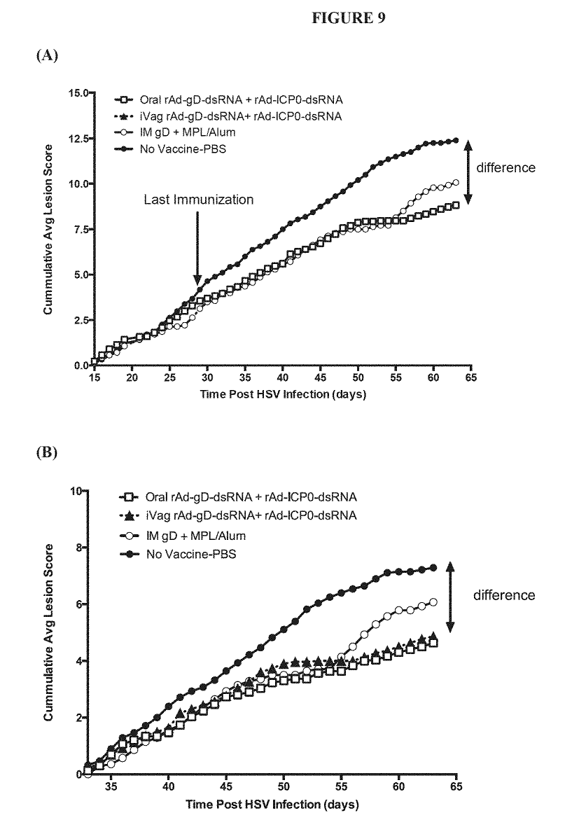

In order to test the contribution of an ICP0 antigen to improve the response, the mixture of rAd-mICP0-dsRNA and rAd-gD-dsRNA (N=15) were compared again to the positive control (N=7) and an unimmunized control group (N=15). Both oral and vaginal delivery methods were tested. The therapeutic model described before was used again where guinea pigs were infected with HSV-2, allowed to recover, and then monitored over time for lesions (FIG. 7). Immunizations were carried out on days 14, 21, and 28. One animal in the unimmunized control group died post HSV-2 infection. FIG. 9A shows that the immunized animals had a lower cumulative average lesion score compared to the untreated animals. Following immunization, the immunized groups had fewer and less severe lesions over time, demonstrating a visible difference by the end of the experiment in the cumulative average lesion score. Focusing on the later time points (day 36 to day 63), it becomes apparent that the animals treated with the combination of rAd-gD-dsRNA and rAd-mICP0-dsRNA have lower cumulative lesion scores than the gD protein +MPL/Alum group (FIG. 9B). In summary, the use of mICP0 and rAd vectors resulted in improved clinical outcome compared to untreated animals and a reduction in lesions compared to the positive control.

### D. Example 4: Prophylactic Effect of HSV-2 Vaccination

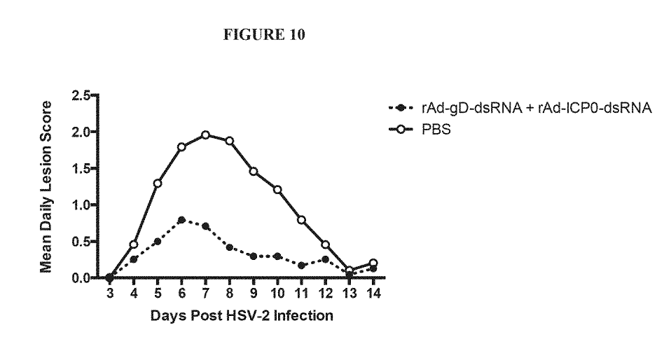

Prevention, rather than treatment of HSV-2 can also theoretically be achieved. We investigated whether oral vaccination with recombinant Ad vectors expressing gD and mICP0 prior to challenge with HSV-2 prevented or reduced clinical symptoms. One group of guinea pigs, group A (n=12) were vaccinated on days 0, 7 and 14, another group of guinea pigs, group B (n=8) were left untreated. Guinea pigs were challenged intravaginally with HSV-2 fourteen days after the final vaccination. Individual guinea pigs were monitored for clinical symptoms and scored daily starting from day 3 post challenge when lesions began to develop. Vaccinated animals had significantly reduced (p=0.02) scores compared to the untreated animals (FIG. 10) suggesting that oral administration of rAd-gD-dsRNA and rAd-mICP0-dsRNA vectors provides significant protection from HSV-2 genital infection.

All publications, patent publications, patents, and Genbank Accession numbers, and websites cited in this specification are herein incorporated by reference in their entireties for all purposes as if each were specifically and individually indicated to be incorporated by reference.

1. An adenoviral vector comprising a promoter operably linked to a polynucleotide encoding an ICP0 antigen, wherein the ICP0 antigen has a mutation in the RING domain of wild type ICP0 from Herpes Simplex Virus-2 (HSV-2) (SEQ ID NO:1).

2. The adenoviral vector of claim 1, wherein the ICP0 antigen comprises at least one fragment of wild type ICP0 polypeptide (SEQ ID NO:1) selected from the group consisting of:
amino acids 83-89;
amino acids 124-150;
amino acids 214-222;
amino acids 636-662;
amino acids 693-701;
amino acids 720-729;
amino acids 741-751; and
amino acids 783-792.

3. The adenoviral vector of claim 1, wherein the ICP0 antigen comprises at least 4 fragments of wild type ICP0 polypeptide (SEQ ID NO:1) selected from the group consisting of:
amino acids 83-89;
amino acids 124-150;
amino acids 214-222;
amino acids 636-662;
amino acids 693-701;
amino acids 720-729;
amino acids 741-751; and
amino acids 783-792.

4. The adenoviral vector of claim 1, wherein the IPC0 antigen is modified in at least one of the conserved amino acids of the RING domain compared to the wild type ICP0 polypeptide (SEQ ID NO:1).

5. The adenoviral vector of claim 1, wherein the ICP0 antigen comprises a polypeptide with at least 90% identity to the sequence of SEQ ID NO:2 or SEQ ID NO:3.

6. The adenoviral vector of claim 1, wherein the ICP0 antigen comprises a polypeptide with the sequence of SEQ ID NO:2 or SEQ ID NO:3.

7. The adenoviral vector of claim 1, further comprising a promoter operably linked to polynucleotide encoding dsRNA.

8. The adenoviral vector of claim 1 further comprising a promoter operably linked to a polynucleotide encoding an HSV-2 capsid, envelope, or tegument protein.

9. The adenoviral vector of claim 8, wherein the HSV-2 protein is glycoprotein B or D.

10. A pharmaceutical composition comprising the adenoviral vector of claim 1 formulated for oral or mucosal administration.

11. A pharmaceutical composition comprising the adenoviral vector of claim 1, further comprising dsRNA or a dsRNA mimetic, wherein the composition is formulated for oral or mucosal administration.

12-13. (canceled)

14. A pharmaceutical composition comprising an ICP0 antigen, wherein the ICP0 antigen has a mutation in the RING domain of wild type ICP0 from Herpes Simplex Virus-2 (HSV-2) (SEQ ID NO:1).

15-16. (canceled)

17. The pharmaceutical composition of claim 14, wherein the IPC0 antigen is modified in at least one of the conserved amino acids of the RING domain compared to the wild type ICP0 polypeptide (SEQ ID NO:1).

18. (canceled)

19. The pharmaceutical composition of claim 14, further comprising a dsRNA or a dsRNA mimetic.

20. (canceled)

21. A method of eliciting an immune response in an individual comprising administering the pharmaceutical composition of claim 14, wherein the immune response includes a cytotoxic T cell response.

22. (canceled)

23. The method of claim 21, wherein the administration is oral or vaginal, and/or wherein the administration is by injection.

24-25. (canceled)

26. The method of claim 21, wherein the administration is monthly, yearly, or episodic as lesions occur.

27. (canceled)

28. A method of reducing an HSV-2 symptom in an individual infected with HSV-2 comprising administering the pharmaceutical composition of claim 14, wherein the HSV-2 symptom is reduced at least 10% compared to the HSV-2 symptom in the individual prior to administration.

29. The method of claim 28, wherein the HSV-2 symptom is selected from frequency of outbreak, severity of lesion, and amount of viral shedding.

30-32. (canceled)

33. A method of vaccinating an uninfected individual against HSV-2 comprising administering the pharmaceutical composition of claim 14 to the individual, thereby protecting the individual from HSV-2 infection.
